(19) United States
(12) Patent Application Publication
(10) Pub. No.: US 2018/0044722 A1
(43) Pub. Date: Feb. 15, 2018

(54) TRI-COLOR PROBES FOR DETECTING MULTIPLE GENE REARRANGEMENTS IN A FISH ASSAY

(71) Applicant: Agilent Technologies, Inc., Santa Clara, CA (US)

(72) Inventors: Michael Ruvolo, San Jose, CA (US); Jimmy Jin, Fremont, CA (US)

(21) Appl. No.: 15/236,264

(22) Filed: Aug. 12, 2016

(51) Int. Cl. C12Q 1/68

(52) U.S. Cl. CPC C12Q 1/6841; C12Q 1/6886; C12Q 2600/156

(57) ABSTRACT

A probe system is provided. In some embodiments, the probe system may comprise: a first labeled probe that hybridizes to one side of a potential translocation breakpoint in a first locus; a second labeled probe that hybridizes to the other side of the potential translocation breakpoint in the first locus; a third labeled probe that hybridizes to one side of a potential translocation breakpoint in a second locus; a fourth labeled probe that hybridizes to the other side of the potential translocation breakpoint in the second locus; and a fifth labeled probe that hybridizes to both sides of the potential translocation breakpoint in either the first locus or the second locus, but not both. The fifth probe is distinguishably labeled from the first, second, third and fourth probes. Methods for detecting a chromosomal rearrangement in the first and second loci using the probe system are also provided.

### BACKGROUND

Fluorescence in situ hybridization (FISH) is a cytogenetic technique that uses fluorescent probes that bind to only those parts of the chromosome with a high degree of sequence complementarity. FISH has long been used to detect chromosomal rearrangements and, because chromosomal rearrangements are quite common in human cancer, FISH is often used to diagnose or assess a malignancy.

Because chromosomal rearrangements are thought to initiate many human cancers, detection of such rearrangements can provide a rationale for treating patients in a particular way. For example, if a non-small-cell lung cancer is associated with a translocation in ALK or ROS1, then the patient can potentially be treated with the same therapy, e.g., crizotinib, which inhibits both ALK and ROS1 (see, generally, Soloman et al J. Clin. Onc. 2015 33: 972-4).

Current FISH methods for detecting particular rearrangements are limited in that they only able to reliably detect such rearrangements one-by-one (see, e.g., Bergethon et al J Clin Oncol 2012 30:863-870). This can be problematic in situations where there is limited sample, which is often the case. Further, performing two separate assays doubles the time to reach a diagnosis, which can lead to a delay in diagnosis and treatment. Multiplex detection of two or more distinct chromosomal rearrangements by FISH would greatly aid in cancer prognostics, diagnostics and theranostics and, as such, are highly desirable.

### SUMMARY

Among other things, a probe system is provided. In some embodiments, the probe system may comprise: a first labeled probe that hybridizes to one side of a potential translocation breakpoint in a first locus; a second labeled probe that hybridizes to the other side of the potential translocation breakpoint in the first locus; a third labeled probe that hybridizes to one side of a potential translocation breakpoint in a second locus; a fourth labeled probe that hybridizes to the other side of the potential translocation breakpoint in the second locus; and a fifth labeled probe that hybridizes to both sides of the potential translocation breakpoint in either the first locus or the second locus, but not both. The first and second probes are distinguishably labeled, the third and fourth probes are distinguishably labeled, and the fifth probe is distinguishably labeled from the first, second, third and fourth probes.

A method for detecting a chromosomal rearrangement using the probe system is also provided. In some embodiments, this method may comprise: (a) hybridizing the probe system with a chromosome in situ to produce to produce a labeled sample; (b) reading the labeled sample to detect hybridization of the labeled probes; and (c) determining whether the sample contains a rearrangement in the first or second locus using the results obtained from the reading step (b).

### BRIEF DESCRIPTION OF THE FIGURES

The patent or application file contains at least one drawing executed in color. Copies of this patent application publication with color drawing(s) will be provided by the U.S. Patent and Trademark Office upon request and payment of the necessary fee.

FIG. 1 schematically illustrates certain features of the present probe system.

FIG. 2 is a diagram showing the position of the constituent probes relative to their target genes, ROS1 (panel A), and ALK (panel B).

FIG. 3 shows results obtained using a ROS1 rearranged sample, showing ROS1 probe signal pattern and the ALK probe signal pattern in the same cell. A) Co-localization of Cy3 and Aqua but no FITC signal indicates that this cell has a ROS1 rearrangement. B) Co-localization of cy3 and FITC signal with lack of Aqua signal indicates non-rearranged ALK in this sample.

FIG. 4 shows results obtained using ALK rearranged sample showing lone red and green signals. The non-rearranged ROS1 gene shows a red/green/blue signal pattern.

### DEFINITIONS

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide” as used herein denotes a single stranded multimer of nucleotide of about 2 to 200 or more, up to about 500 nucleotides or more. Oligonucleotides may be synthetic or may be made enzymatically, and, in some embodiments, are less than 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200 nucleotides in length, for example.

The term “sequence-specific oligonucleotide” as used herein refers to an oligonucleotide that only binds to a single site in a haploid genome. In certain embodiments, a “sequence-specific” oligonucleotide may hybridize to a complementary nucleotide sequence that is unique in a sample under study.

The term “complementary” as used herein refers to a nucleotide sequence that base-pairs by non-covalent bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is fully complementary to a target of interest such that every nucleotide in the sequence is complementary to every nucleotide in the target nucleic acid in the corresponding positions. In certain cases, a nucleotide sequence may be partially complementary to a target, in which not all nucleotide is complementary to every nucleotide in the target nucleic acid in all the corresponding positions.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “hybridization” refers to the specific binding of a nucleic acid to a complementary nucleic acid via Watson-Crick base pairing. Accordingly, the term “in situ hybridization” refers to specific binding of a nucleic acid probe to a metaphase or interphase chromosome.

The terms “hybridizing” and “binding”, with respect to nucleic acids, are used interchangeably.

The terms “plurality”, “set” or “population” are used interchangeably to mean at least 2, at least 10, at least 100, at least 500, at least 1000, at least 10,000, at least 100,000, at least 1000,000, at least 10,000,000 or more.

The term “chromosomal rearrangement,” as used herein, refers to an event where one or more parts of a chromosome are rearranged within a single chromosome or between chromosomes. In certain cases, a chromosomal rearrangement may reflect an abnormality in chromosome structure. A chromosomal rearrangement may be an inversion, a deletion, an insertion or a translocation, for example.

The term “potential translocation breakpoint” refers to a translocation breakpoint that may or may not be present in the sample under study. In some cases, a potential translocation breakpoint may be known from other studies and may be correlated with a disease or treatment.

The terms “one side” and “the other side”, in the context of a potential translocation breakpoint refer, to regions that are on opposite sides of the site of a potential translocation breakpoint. In some instances “one side” and “the other side” may be referred to as the first side and the second side, where the first and second sides are on opposite sides of a potential translocation breakpoint. If a probe binds to one side or the other side of a potential translocation breakpoint, then the probe may bind to sequences that are less than 10 MB, e.g., less than 5 MB, less than 1 MB, less than 500 kb or less than 100 kb away from the potential translocation breakpoint, although probes that bind to sequence that are greater than 10 MB away from the potential translocation breakpoint may be used in some circumstances.

The term “locus” refers to a contiguous length of nucleotides in a genome of an organism. A chromosomal region may be in the range of 100 bases in length to an entire chromosome, e.g., 100 kb to 10 MB for example. In some embodiments, a locus may be 100 bp to 1 MB, e.g., 1 kb to 1 Mb, in length.

The terms “first locus” and “second locus” refer to sequences that are either unlinked (i.e., on different chromosomes) or sufficiently distanced on the same chromosomes (e.g., on different chromosome arms) that they can be resolved by FISH.

The term “in situ” in the context of an in situ hybridization refers to conditions that allow hybridization of a nucleic acid to a complementary nucleic acid in an interphase or metaphase cell that contains relatively intact chromosomes (some fragmentation occurs during the process). Suitable in situ hybridization conditions may include both hybridization conditions and optional wash conditions, which include temperature, concentration of denaturing reagents, salts, incubation time, etc. Such conditions are known in the art. A “test cell” may contain a metaphase or interphase chromosome, where such a chromosome contains a centromere, a long arm containing a telomere and a short arm containing a telomere. A test chromosome may contain an inversion, translocation, deletion insertion, or other rearrangement relative to a reference chromosome that does not have a translocation. The test cells from the sample under study.

A “binding pattern” refers to the pattern of binding of a set of labeled probes to the chromosomes of a cell, in situ.

The term “ALK” refers to the gene that encodes anaplastic lymphoma kinase (also also known as ALK tyrosine kinase receptor or CD246 (cluster of differentiation 246)); see Morris et al Science 1994 263: 1281-4 and NCBI Entrez Gene ID: 238. This gene encodes a receptor tyrosine kinase, which belongs to the insulin receptor superfamily. This protein comprises an extracellular domain, an hydrophobic stretch corresponding to a single pass transmembrane region, and an intracellular kinase domain. It plays an important role in the development of the brain and exerts its effects on specific neurons in the nervous system. This gene has been found to be rearranged, mutated, or amplified in a series of tumours including anaplastic large cell lymphomas, neuroblastoma, and non-small cell lung cancer. The chromosomal rearrangements are the most common genetic alterations in this gene, which result in creation of multiple fusion genes in tumourigenesis, including ALK (chromosome 2)/EML4 (chromosome 2), ALK/RANBP2 (chromosome 2), ALK/ATIC (chromosome 2), ALK/TFG (chromosome 3), ALK/NPM1 (chromosome 5), ALK/SQSTM1 (chromosome 5), ALK/KIFSB (chromosome 10), ALK/CLTC (chromosome 17), ALK/TPM4 (chromosome 19), and ALK/MSN (chromosome X). The gene is located in human chromosome 2 at chr 2: 29.19-29.92 Mb.

The term “ROS1” refers to the gene that encodes the proto-oncogene tyrosine-protein kinase ROS (also known as ROS1, MCF3, ROS and c-ros-1) (see, e.g., Galland et al 1992 Cytogenetics and Cell Genetics 60 (2): 114-6 and NCBI Entrez Gene ID: 6098). Gene rearrangements involving the ROS1 gene were first detected in glioblastoma tumors and cell lines. In 2007 a ROS1 rearrangement was identified in a cell line derived from a lung adenocarcinoma patient. Since that discovery, multiple studies have demonstrated an incidence of approximately 1% in lung cancers. The gene is located in human chromosome 2 at Chr 6: 117.29-117.43 Mb.

### DESCRIPTION OF EXEMPLARY EMBODIMENTS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Probe Systems

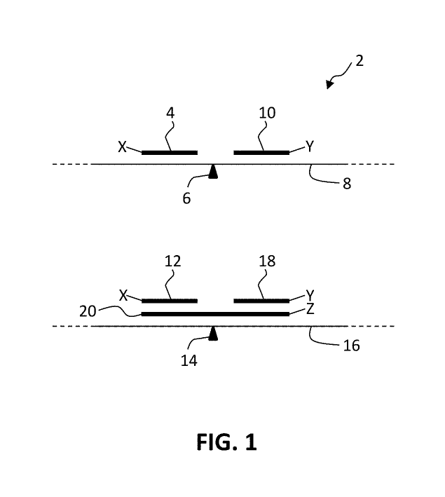

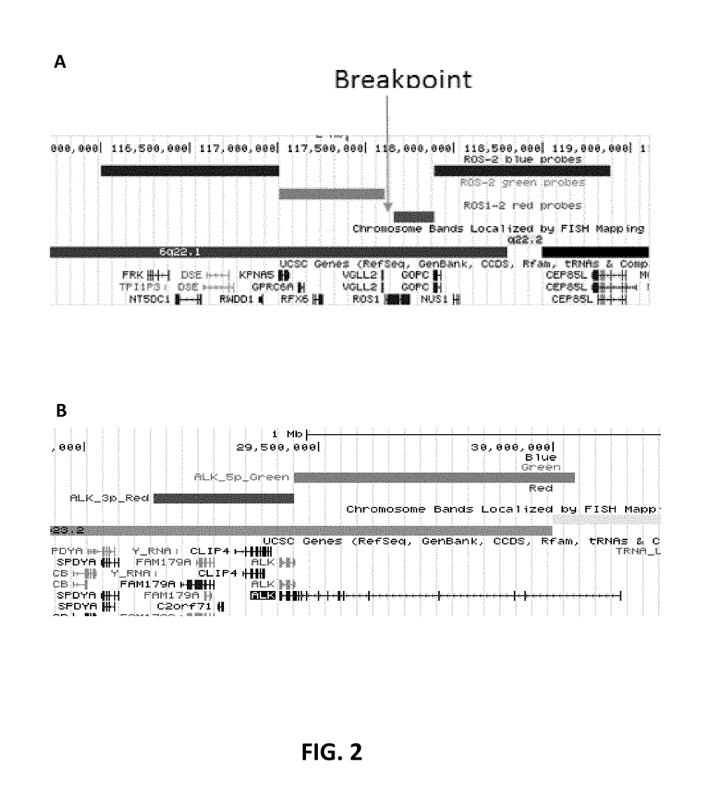

With reference to FIG. 1, probe system 2 may comprise: a) a first labeled probe 4 that hybridizes to one side of a potential translocation breakpoint 6 in a first locus 8; b) a second labeled probe 10 that hybridizes to the other side of potential translocation breakpoint 6 in first locus 8; c) a third labeled probe 12 that hybridizes to one side of a potential translocation breakpoint 14 in a second locus 16; d) a fourth labeled probe 18 that hybridizes to the other side of potential translocation breakpoint 14 in second locus 16; and e) a fifth labeled probe 20 that hybridizes to both sides of the potential translocation breakpoint in either the first locus or the second locus, but not both (i.e., both sides of potential translocation breakpoint 6 or 14 but not both, where: i. first and second probes 4 and 10 are distinguishably labeled (e.g., labeled with distinguishable fluorophores X and Y, respectively); ii. third and fourth probes 12 and 18 are distinguishably labeled (e.g., labeled with distinguishable fluorophores X and Y, respectively); and iii. fifth probe 20 is distinguishably labeled from the first, second, third and fourth probes (e.g., labeled with distinguishable fluorophore Z). In FIG. 1 fifth labeled probe 20 is shown as hybridizing to both sides of the potential translocation breakpoint in the first locus 8. Alternatively, fifth labeled probe 20 can hybridize to both sides of the potential translocation breakpoint in the second locus 16. In the implementation shown in FIG. 1, the sequence targeted by the fifth probe overlaps with the other probes that hybridize to that locus (i.e., the first and second, or the third and forth probes). In some cases, and as illustrated in the example shown in FIG. 2, panel A, the sequence targeted by the fifth probe do not overlap with the other probes that hybridize to that locus (i.e., the first and second, or the third and forth probes). In these embodiments, the fifth labeled probe that hybridizes to both sides of the potential translocation breakpoint in either locus (e.g., ALK or ROS1), but not both; wherein: i. the first and second probes are distinguishably labeled; ii. the third and fourth probes are distinguishably labeled; and iii. the fifth probe is distinguishably labeled from the first, second, third and fourth probes, and the sequence to which the fifth probe hybridizes does not overlap with the sequences to which the first, second, third and fourth probes hybridize.

As would be apparent, the first and second loci may be regions in mammalian chromosomes, particularly human chromosomes. In some embodiments, the first locus and the second locus are genes, e.g., genes selected from ALK, RET, ROS1, C-MYC, cyclin D1, BCL-2, PAX8, ETO, ABL1, PML, TEL, JAK, API-2, FLI1 and FUS, all of which contain potential translocation breakpoints that are associated with various cancers. In particular embodiments of the first locus may be ALK and the second locus may be ROS1. In these embodiments, the probe system may comprise: (a) a first labeled probe that hybridizes to one side of a potential translocation breakpoint in ALK; (b) a second labeled probe that hybridizes to the other side of the potential translocation breakpoint in ALK; (c) a third labeled probe that hybridizes to one side of a potential translocation breakpoint in ROS1; (d) a fourth labeled probe that hybridizes to the other side of the potential translocation breakpoint ROS1; and (e) a fifth labeled probe that hybridizes to both sides of the potential translocation breakpoint in either ALK or ROS1, but not both, where: i. the first and second probes are distinguishably labeled, ii. the third and fourth probes are distinguishably labeled; and iii. the fifth probe is distinguishably labeled from the first, second, third and fourth probes.

Approximately 60% of anaplastic large-cell lymphomas (ALCLs) are associated with an ALK translocation. In many of these translocations, the translocation creates a fusion gene consisting of the 3′ half of the ALK gene from chromosome 2 and 5′ portion of the nucleophosmin (NPM) gene from chromosome 5. This product of the NPM-ALK fusion gene is oncogenic. In other translocations, the 3′ half of ALK is fused to the 5′ sequence of TPM3 gene, encoding fortropomyosin 3. In rare cases, ALK is fused to other 5′ fusion partners, such as TFG, ATIC, CLTC1, TPM4, MSN, ALO17, MYH9. The EML4 translocations are responsible for approximately 3-5% of non-small-cell lung cancer (NSCLC). The vast majority of cases are adenocarcinomas. ALK lung cancers are found in patients of all ages, although on average these patients tend to be younger. ALK lung cancers are more common in light cigarette smokers or nonsmokers, but a significant number of patients with this disease are current or former cigarette smokers. EML4-ALK-rearrangement in NSCLC is exclusive and not found in EGFR- or KRAS-mutated tumors. ALK translocations are also associated with familial cases of neuroblastoma, inflammatory myofibroblastic tumor, adult and pediatric renal cell carcinomas, esophageal squamous cell carcinoma, breast cancer, notably the inflammatory subtype, colonic adenocarcinoma, glioblastoma multiforme and anaplastic thyroid cancer.

Genetic changes in ROS1, such as gene rearrangements, create oncogenes that can also lead to cancer (Stumpfova and Janne, Clin Cancer Res. 2012 18: 4222-4). ROS1 was discovered in NSCLC patients in the form of a fusion protein (6 different partners for ROS1) and is found in approximately 2% of patients with NSCLC (J Clin Oncol. 2012 Mar. 10; 30(8):863-70). Two other ROS1 gene rearrangements have been detected in a variety of other cancers, including glioblastoma multiforme, cholangiocarcinoma, ovarian cancer, gastric adenocarcinoma, colorectal cancer, inflammatory myofibroblastic tumor, angiosarcoma, and epitheloid hemangioendothelioma. ROS1 gene rearrangements create fusion proteins with constitutively active kinase domains that activate downstream signaling pathways leading to oncogenic properties in cells, including uncontrolled proliferation and resistance to cell death with prolonged tumor cell survival. These pathways include Ras-ERK for cellular proliferation and the JAK-STAT and PI3K/AKT pathways, which regulate cell survival (anti-apoptosis) and proliferation. ROS1 fusion proteins may also activate the mTOR pathway, which is critical for the regulation of protein translation. Cancers that have these pathways activated tend to be more aggressive, with invasion and metastasis leading to poor survival of the patients.

In some embodiments, the first and third probes are labeled with a first fluorophore (i.e., the same fluorophore). The second and fourth probes can be labeled with a second fluorophore (a fluorophore that is distinguishable from the first fluorophore). The fifth probe should be distinguishably labeled from the first, second, third and fourth probes, e.g., using a third distinguishable fluorophore. In some embodiments, the first and third probes are labeled with a first fluorophore, the second and fourth probes are labeled with a second fluorophore; and the fifth probe is labeled with a third fluorophore. As used herein, the the term “distinguishably labeled” means that the labels can be separately detected, even if they are at the same location. As such, the fluorophores used should be chosen so that they are distinguishable, i.e., independently detectable, from one another, meaning that the labels can be independently detected and measured, even when the labels are mixed. In other words, the presence of each label should be separately determinable, even when the labels are co-located.

Suitable sets of distinguishable labels include, but are not limited to RD1, FITC, and EDC; PerCP, phycoerythrin, and fluorescein isothiocyanate; Fluorescein, Cy3 and Cy5; and rhodamine, fluorescein, and Cyanine-5, and equivalents thereof. Specific fluorescent dyes of interest include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g., Cy3, Cy5 and Cy7 dyes; coumarins, e.g., umbelliferone; benzimide dyes, e.g. Hoechst 33258; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, Napthofluorescein, Texas Red, Cy3, and Cy5, etc. Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 and TOPRO3 (Molecular Probes, Eugene, Oreg.). Further suitable distinguishable detectable labels may be found in Kricka et al. (Ann Clin Biochem. 39:114-29, 2002), Ried et al. (Proc. Natl. Acad. Sci. 1992: 89: 1388-1392) and Tanke et al. (Eur. J. Hum. Genet. 1999 7:2-11) and others.

The region to which each of the probes hybridizes may be at least 5 kb in length, e.g., in the range of 5 kb to 100 kb, 100 kb to 500 kb, 500 kb to 1 Mb, 1 Mb to 5 Mb, 5 Mb to 10 Mb or 10 Mb to 50 Mb, 10 kb to 1 mb, or 10 kb to 500 kb in length, etc. In some embodiments, each probe may comprise a plurality of labeled fragments of nucleic acid, e.g., at least 50, at least 100, at least 500 or at least 1,000 fragments of nucleic acid. In some embodiments, the probes may comprise labeled double-stranded nucleic acid. The probes can be made using any suitable method, e.g., by random-priming or nick translation of bacterial artificial chromosomes, fosmids, or other sources of DNA. In some embodiments, the probes may be made using methods descried in Yamada et al (Cytogenet Genome Res 2011; 132:248-254) and U.S. Pat. No. 8,034,917), which involve synthesizing a high complexity library of long oligonucleotides (>150 mers) that target to only the most informative elements, amplifying probes from the library, and labeling those probes during or after amplification. In some cases, the amplification may be done in the presence of a labeled nucleotide. The binding sites for the molecules of a probe may be tiled across a region such that there is an overlap between adjacent binding sites (such that there is, for example, a 10% to 90% overlap between the probe molecules, when bound) or they may be tiled end-to-end such that the 5′ end of one binding site is next to the 3′ end of the binding site. In another embodiment, the binding sites for the molecules of a probe may be separated and interspersed within the chromosomal region. Methods may be used for labeling the probes are Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). In some embodiments, FISH probes can be labeled with biotin using the Universal Linkage System (ULS.™., KREATECH Diagnostics; van Gijlswijk et al Universal Linkage System: versatile nucleic acid labeling technique Expert Rev. Mol. Diagn. 2001 1:81-91), which is based on the stable binding properties of platinum (II) to nucleic acids.

Method of Sample Analysis

Also provided is a method of sample analysis. This method involves (a) hybridizing the probe system with a chromosome in situ to produce to produce a labeled sample; (b) reading the labeled sample to detect hybridization of the labeled probes, e.g., using fluorescence microscope equipped with an appropriate filter for each fluorophore, or by using triple band-pass filter sets to observe multiple fluorophores (see, e.g., U.S. Pat. No. 5,776,688); and (c) determining whether the sample contains a rearrangement in the first locus or the second locus using the results obtained from the reading step (b). As would be apparent, if the first and second loci are ALK and ROS1, the method would comprise (c) determining whether the sample contains a rearrangement in ALK or ROS1 using the results obtained from the reading step (b).

A rearrangement can be determined by examining the images produced in the reading step. Specifically, if the fifth probe hybridizes to both sides of the potential translocation breakpoint in the first locus, then:

a) in a “normal” cell (a cell that does not have a rearrangement in the first locus or the second locus): i. the first and second probes co-localize with each other and with the fifth probe, and ii. the third and fourth probes co-localize with each other but not with the fifth probe;

b) in a cell that contains a translocation in the first locus: i. the first and second probes co-localize with the fifth probe but not with each other, and ii. the third and fourth probes co-localize with each other but not with the fifth probe; and

c) in a cell that contains a translocation in the second locus: i. the first and second probes co-localize with the fifth probe and with each other, and ii. the third and fourth probes do not co-localize with each other or the fifth probe.

Likewise, if the fifth probe hybridizes to both sides of the potential translocation breakpoint in the second locus, then:

a) in a “normal” cell (a cell that does not have a rearrangement in the first locus or the second locus): i. the first and second probes co-localize with each other but not with the fifth probe, and ii. the third and fourth probes co-localize with each other and with the fifth probe;

b) in a cell that contains a translocation in the first locus: i. the first and second probes do not co-localize with each other or with the fifth probe, and ii. the third and fourth probes co-localize with the fifth probe and with each other; and

c) in a cell that contains a translocation in the second locus: i. the first and second probes co-localize with each other but not with the fifth probe, and ii. the third and fourth probes co-localize with the fifth probe but not with each other.

Given the principle of this method, higher complexity probe systems, e.g., probe systems that contain four or even five distinguishable fluorophores can be designed and implemented.

In situ hybridization methods, which generally involve mounting cells to a support, fixing and permeabilizing the cells, hybridizing probes to the chromosomes in the cells in situ, washing away unbound cells and imaging the labeled cells using fluorescence microscopy, are well known in the art, as such, given the present description the method may be adapted from known protocols. See, e.g., Jin, Journal of Clinical Laboratory Analysis 1997 11 (1): 2-9. Indeed, given the present description some embodiments of the method may be adapted from clinically approved methodology for assessing rearrangements in ALK (see, e.g., Gao et al J. Thorac. Oncol. 2015 10:1648-52; Conde et al PLoS One 2014 9:e107200, Kim et al Transl. Lung Cancer Res. 2015 4: 149-55 and Teixidó et al Transl. Lung Cancer Res. 2014 3:70-4).

In some embodiments, the sample may be ready in three channels corresponding to the labels used to produce a plurality of images of the sample. The images produced by the method may be viewed side-by-side or, in some embodiments, the images may be superimposed or combined. In some cases, the images may be in color, where the colors used in the images may correspond to the labels used.

In some embodiments, the method may further comprise analyzing, comparing or overlaying, at least two of the images. In some embodiments, the method may further comprise overlaying all of the images to produce an image showing the pattern of binding of all of the probes to the sample. The image analysis module used may transform the signals from each fluorophore to produce a plurality of false color images. The image analysis module may overlay the plurality of false color images (e.g., superimpose the false colors at each pixel) to obtain a multiplexed false color image. Multiple images (e.g., unweighted or weighted) may be transformed into a single false color, e.g., so as to represent a biological feature of interest characterized by the binding of a specific probe. False colors may be assigned to specific probes or combinations of probes, based on manual input from the user. The image analysis module may further be configured to adjust (e.g., normalize) the intensity and/or contrast of signal intensities or false colors, to perform a convolution operation (such as blurring or sharpening of the intensities or false colors), or perform any other suitable operations to enhance the image. The image analysis module may perform any of the above operations to align pixels obtained from successive images and/or to blur or smooth intensities or false colors across pixels obtained from successive images.

The image analysis method may be implemented on a computer. In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer, which runs the program, and returns an output to the user.

Any type of cell can be analyzed using this method. In certain instances, the sample analyzed may be a fresh or embedded (e.g., FFPE embedded) tissue biopsy obtained from a patient. Biopsies of interest include both tumor and non-neoplastic biopsies of skin (melanomas, carcinomas, etc.), soft tissue, bone, breast, colon, liver, kidney, adrenal gland, gastrointestinal tissue, pancreas, gall bladder, salivary gland, cervical, ovary, uterus, testis, prostate, lung, thymus, thyroid, parathyroid, pituitary (adenomas, etc.), brain, spinal cord, ocular tissue, nerve, and skeletal muscle, etc.

In any embodiment, data (e.g., images of the cells) can be forwarded to a “remote location,” where “remote location” means a location other than the location at which the data is produced. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but be separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or include email transmissions and information recorded on websites and the like. In certain embodiments, one or more images produced by the method may be analyzed by an MD or other qualified medical professional, and a report based on the results of the analysis of the image may be forwarded to the patient from which the sample was obtained.

Utility

The present method may be employed in a variety of diagnostic, drug discovery, and research applications that include, but are not limited to, diagnosis or monitoring of a disease or condition (where a rearrangement in the first or second locus may be marker for the disease or condition), discovery of drug targets (where a rearrangement in the first or second locus may be targeted for drug therapy), drug screening (where the effects of a drug are monitored by a rearrangement in the first or second locus is), determining drug susceptibility (where drug susceptibility is associated with a rearrangement in the first or second locus is) and basic research (where is it desirable to identify a rearrangement in the first or second locus). As such, in some embodiments, the method may comprising providing a diagnosis, theranosis or prognosis if the sample contains the rearrangement.

In some cases, the method described herein can be used to determine a treatment plan for a patient. The presence or absence of a rearrangement in the first or second locus may indicate that a patient is responsive to or refractory to a particular therapy. For example, a presence or absence of one or more biomarkers may indicate that a disease is refractory to a specific therapy and an alternative therapy can be administered.

In embodiments in which the first and second loci are ALK and ROS1, respectively, a rearrangement in either locus may indicate that a patient should be treated with crizotinib or another kinase inhibitor. In these embodiments, if a rearrangement in ALK or ROS1 is identified, then a healthcare professional (e.g., an MD or the like) may provide a recommendation for treatment by crizotinib. Crizotinib is an anti-cancer drug acting that inhibits ALK (anaplastic lymphoma kinase) and ROS1 (c-ros oncogene 1) (see, e.g., Forde Expert Opin. Pharmacother. 2012 13: 1195-201; Roberts Biologics 2013 7: 91-101; Sahu et al South Asian J. Cancer 2: 91-7) that has been approved for treatment of some non-small cell lung carcinoma (NSCLC) in the US and other countries, and undergoing clinical trials testing its safety and efficacy in anaplastic large cell lymphoma, neuroblastoma, and other advanced solid tumors in both adults and children.

### ALTERNATIVE EMBODIMENTS

In some alternative embodiments, the system does not contain the fifth probe. In these embodiments, the probe system may comprise: (a) a first labeled probe that hybridizes to one side of a potential translocation breakpoint in a first locus (e.g., ALK); (b) a second labeled probe that hybridizes to the other side of the potential translocation breakpoint in the first locus (e.g., ALK); (c) a third labeled probe that hybridizes to one side of a potential translocation breakpoint in a second locus (e.g., ROS1); and (d) a fourth labeled probe that hybridizes to the other side of the potential translocation breakpoint in the second locus (e.g.,) ROS1; wherein: i. the first and second probes are distinguishably labeled; and ii. the third and fourth probes are distinguishably labeled. In use of such a probe system, if a chromosome translocation is identified, one would have to perform follow-up work to identify which locus has the translocation.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

### EMBODIMENTS

### Embodiment 1

A probe system comprising: a) a first labeled probe that hybridizes to one side of a potential translocation breakpoint in a first locus; b) a second labeled probe that hybridizes to the other side of the potential translocation breakpoint in the first locus; c) a third labeled probe that hybridizes to one side of a potential translocation breakpoint in a second locus; d) a fourth labeled probe that hybridizes to the other side of the potential translocation breakpoint in the second locus; and e) a fifth labeled probe that hybridizes to both sides of the potential translocation breakpoint in either the first locus or the second locus, but not both; wherein: i. the first and second probes are distinguishably labeled; ii. the third and fourth probes are distinguishably labeled; and iii. the fifth probe is distinguishably labeled from the first, second, third and fourth probes.

### Embodiment 2

The method of embodiment 1, where the first and third probes are labeled with a first fluorophore.

### Embodiment 3

The method of any prior embodiment, wherein the second and fourth probes are labeled with a second fluorophore.

### Embodiment 4

The method of any prior embodiment, wherein: the first and third probes are labeled with a first fluorophore; the second and fourth probes are labeled with a second fluorophore; and the fifth probe is labeled with a third fluorophore

### Embodiment 5

The method of any prior embodiment, wherein each of the probes spans at least 10 kb.

### Embodiment 6

The method of any prior embodiment, wherein each probe comprises a plurality of labeled fragments of nucleic acid.

### Embodiment 7

The method of any prior embodiment, wherein each probe comprises labeled double-stranded nucleic acid.

### Embodiment 8

The method of any prior embodiment, wherein the first locus and the second locus are genes.

### Embodiment 9

The method of any prior embodiment, wherein the first locus is ALK and the second locus is ROS1.

### Embodiment 10

A method of sample analysis, comprising: (a) in situ hybridizing a cell comprising chromosomes with a probe system of claim 1 to produce to produce a labeled sample; (b) reading the labeled sample to detect hybridization of the labeled probes; and (c) determining whether the sample contains a rearrangement in the first locus or the second locus using the results obtained from the reading step (b).

### Embodiment 11

The method of embodiment 10, wherein the reading is done by fluorescence microscopy.

### Embodiment 12

The method any prior method embodiment, wherein the cell is a mammalian cell.

### Embodiment 13

The method any prior method embodiment, wherein the wherein the first locus is ALK and the second locus is ROS1.

### Embodiment 14

The method any prior method embodiment, further comprising providing a diagnosis, theranosis or prognosis if the sample contains the rearrangement.

In order to further illustrate the present invention, the following specific examples are given with the understanding that they are being offered to illustrate the present invention and should not be construed in any way as limiting its scope.

### EXAMPLE

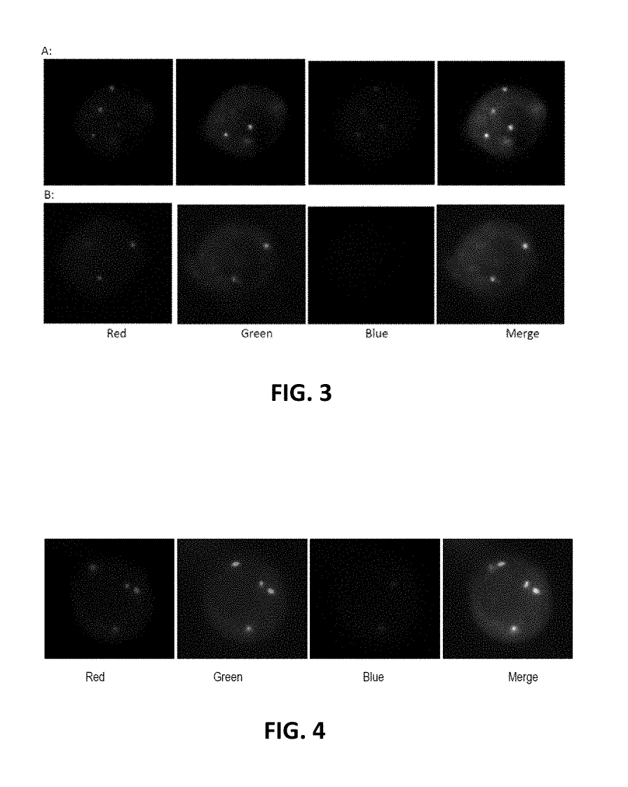

The purpose of the study was to demonstrate that samples harboring either an ALK or ROS1 gene rearrangement could be distinguished (i.e. each would have a unique fluorescent signal pattern) using a single DNA FISH probe assay. To do this, the individual constituent probes described in Table 1 and FIG. 2 were generated and combined to make the final probe targeting both the ALK and ROS1 loci. For samples rearranged for ROS1, at least one red/blue and/or green/blue signal pair will be seen (FIG. 3). In samples rearranged for ALK, and least one red and/or green signal will be observed (FIG. 4).

Long oligonucleotide libraries targeting the regions outlined in table 1 were designed and synthesized. The oligonucleotides were used as a template to generate the labeled constituent probes using polymerase chain reaction (PCR) to incorporate the red, green, or blue fluorophore. The labeled constituent probes were combined and mixed with FISH hybridization buffer to generate the final probe. This probe was hybridized to cell lines harboring either an ALK or ROS1 gene rearrangement. The hybridized samples were visualized using an epi-fluorescent microscope fitted with Cy3 (Red), FITC (Green), and Aqua (Blue) filters. Images depicting the signal pattern observed on the rearranged samples were captured (FIGS. 3 and 4).

TABLE 1
Genomic region targeted by each of the constituent probes.
Coordinates are based on the Human Genome Build 19 (Hg19).

| Constituent | |
|---|---|
| Probe | Region |
| ROS1 3′ Red | chr6: 117320499-117609677 |
| ROS1 5′ Green | chr6: 117747132-118252359 |
| ROS1 3′ Blue | chr6: 116510473-117609523 |
| ROS1 5′ Blue | chr6: 117747013-118899513 |
| ALK 3′ Red | chr2: 29146786-29446528 |
| ALK 5′Green | chr2: 29446949-30045655 |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A probe system comprising:
(a) a first labeled probe that hybridizes to one side of a potential translocation breakpoint in ALK;
(b) a second labeled probe that hybridizes to the other side of the potential translocation breakpoint in ALK;
(c) a third labeled probe that hybridizes to one side of a potential translocation breakpoint in ROS1;
(d) a fourth labeled probe that hybridizes to the other side of the potential translocation breakpoint in ROS1; and
(e) a fifth labeled probe that hybridizes to both sides of the potential translocation breakpoint in either ALK or ROS1, but not both;
wherein: i. the first and second probes are distinguishably labeled;
ii. the third and fourth probes are distinguishably labeled; and
iii. the fifth probe is distinguishably labeled from the first, second, third and fourth probes.

2. The probe system of claim 1, where the first and third probes are labeled with a first fluorophore.

3. The probe system of claim 1, wherein the second and fourth probes are labeled with a second fluorophore.

4. The probe system of claim 1, wherein:
the first and third probes are labeled with a first fluorophore;
the second and fourth probes are labeled with a second fluorophore; and
the fifth probe is labeled with a third fluorophore

5. The probe system of claim 1, wherein each of the probes of (a)-(e) spans at least 10 kb.

6. The probe system of claim 1, wherein each probe comprises a plurality of labeled fragments of nucleic acid.

7. The method of claim 1, wherein each probe comprises labeled double-stranded nucleic acid.

8. A method of sample analysis, comprising:
(a) hybridizing a probe system of claim 1 with a chromosome in situ to produce a labeled sample;
(b) reading the labeled sample to detect hybridization of the labeled probes; and
(c) determining whether the sample contains a rearrangement in ALK or ROS1 using the results obtained from the reading step (b).

9. The method of claim 8, wherein the reading is done by fluorescence microscopy.

10. The method of claim 8, wherein the cell is a mammalian cell.

11. The method of claim 8, further comprising providing a diagnosis, theranosis or prognosis if the sample contains the rearrangement.

12. The method of claim 8, further comprising providing a recommendation for treatment by crizotinib the sample contains the rearrangement.
